(19) United States
(12) Patent Application Publication
(10) Pub. No.: US 2006/0030808 A1
(43) Pub. Date: Feb. 9, 2006

(54) Liquid bandage and tissue sealant

(75) Inventor: John P. Kennedy, Sarasota, FL (US)

Correspondence Address: Law Offices of John D. Gugliotta, PE, Esq., 202 Delaware Building, 137 South Main Street, Akron, OH, 44308, US

(73) Assignee: ASO CORPORATION

(21) Appl. No.: 10/914,069

(22) Filed: Aug. 9, 2004

(51) Int. Cl. A61F 15/00

(52) U.S. Cl. 602/41

(57) ABSTRACT

A fluid composition is provided which forms in situ a protective or preventative covering, closure or seal for superficial and non-superficial cuts, scrapes, abrasions, burns, exposed tissues, open wounds and the like. The fluid composition is applied as a fluid-like, coatable formulation ultimately creating a flexible, protective seal on and around the affected area which lowers the probability of contamination and promotes fast healing.

### RELATED APPLICATIONS

There are no previously filed, nor currently any co-pending applications, anywhere in the world.

### BACKGROUND OF THE INVENTION

1. Field of the Invention

The present invention relates generally to compositions for forming covering elements suitable for use as a bandage, and more particularly, to a fluid-like, coatable composition adapted for use as a bandage, skin protectant, tissue sealant, and/or skin closure.

2. Description of the Related Art

There has been a long felt need for improving consumer treatment of minor cuts, scrapes, burns and the like. Focus is directed on providing a bandage and skin protectant for infection prevention and pain reduction which is simple to apply, flexes during movement, and adheres better than conventional adhesive bandages. For wounds or applications that are not superficial, tissue sealant and skin closure applications have received considerable attention.

In addition, the innovative bandage, skin protectant, tissue sealant, or skin closure should be applied as a fluid-like, coatable composition creating a flexible, breathable seal, on and around the affected area, which promotes fast healing.

Further, the innovative bandage, skin protectant, tissue sealant, or skin closure should adhere to hard-to-reach areas more readily than ordinary bandages until sloughed from the tissue during the natural cell cyclical regeneration process.

Accordingly, there is a need for a fluid-like, coatable composition adapted to form, in situ, a protective covering and sealing element which functions as a liquid bandage, skin protectant, tissue sealant, and skin closure. The development of the liquid bandage and tissue sealant fulfills this need.

A search of the prior art did not disclose any patents that read directly on the claims of the instant invention; however, the following references were considered related.

The following patents disclose combinations of alky siloxy siloxane-containing polymers admixed with liquid polydimethylsiloxanes which provide liquid coating material for forming films which act as conformable bandages:

U.S. Pat. No. 4,987,893, issued in the name of Salamone et al.; and U.S. Pat. No. 5,103,812, issued in the name of Salamone et al.

U.S. Pat. No. 6,627,216 B2, issued in the name of Brandt et al. discloses a fluid composition suitable for in situ forming and adhering a touch-dry, non-tacky covering element onto a surface.

U.S. Pat. No. 6,512,023 B1, issued in the name of Malofsky et al. discloses a stabilized monomer adhesive composition.

U.S. Pat. No. 6,605,667 B1, issued in the name of Badejo et al. discloses antioxidant enriched adhesive compositions and storage containers therefor.

U.S. Pat. No. 6,667,031 B2, issued in the name of Azevedo discloses a method for curing cyanoacrylate adhesives.

U.S. Pat. Nos. 5,981,621 and 6,565,840 B1, issued in the name of Clark et al. disclose monomeric compositions effective as wound closure devices.

U.S. Pat. No. 6,183,593, issued in the name of Narang et al. discloses an adhesive composition that contains cyanoacrylate monomers and cyclic or alkyl- or phenyl-terminated linear polydimethylsiloxane being particularly useful in medical applications as an alternate or an adjunct to surgical sutures and/or staples in wound closure, as well as for covering and protecting surface wounds.

U.S. Pat. No. 6,143,805, issued in the name of Hickey et al. discloses a method for sterilizing a liquid adhesive composition useful as biomedical adhesives and sealants wherein such method includes subjecting the composition to electron beam irradiation while enclosed in a container.

### SUMMARY OF THE INVENTION

Therefore, it is an object of the present invention to provide a fluid composition adapted to form a protective or preventative covering or bandage for superficial minor cuts, scrapes, abrasions, burns and the like.

It is another object of the present invention to provide a fluid composition adapted to form a seal on non-superficial tissues or to close open tissues exceeding minor cuts, scrapes and abrasions.

It is another object of the present invention to provide a fluid composition in the form of a liquid, viscous liquid or gel.

It is another object of the present invention to provide a fluid composition formulated such that the combination of short and long alkyl chain monomers and/or plasticizers ensure flexibility of the resulting polymerized protective coating or seal.

It is another object of the present invention to provide a fluid composition formulated such that the viscosity may be optimized for the intended application.

It is another object of the present invention to provide a fluid composition formulated such that a stabilizer may be incorporated to enhance stability of the fluid composition.

It is another object of the present invention to provide a fluid composition packaged such that multiple applications may be dispensed out of the same container.

It is another object of the present invention to provide a fluid composition packaged such that a trial quantity or limited number of applications may be dispensed.

Briefly described according to one embodiment of the present invention, a fluid composition comprised of a coatable composition is adapted to form, in situ, a protective superimposing element, seal or covering element.

The coatable composition is defined as an effective amount of a flexible adherence component, either long chain monomer and/or plasticized short to moderate chain monomers, in order that the covering element flexes on the desired substrate yet is not undesirably tacky or too soft.

A particularly beneficial, as well as advantageous, class of adherent materials suitable for use as the adherent component is the class of cyanocrylate monomers, preferably a blend of cyanoacrylate monomers in a formulation of synergistic components. A preferred cyanoacrylate monomer component, or blend of components [monomer(s) A], is derived from monomers comprising, based upon total weight of the formulation, approximately 10 to 100 weight percent. The A monomer is selected from at least one alkyl radical containing approximately 1 to 5 carbon atoms in the alkyl group. Representative examples of an alkyl cycanoacrylate monomer include methyl, ethyl, n-butyl, isobutyl, n-pentyl, 2-pentyl or 3-pentyl cyanoacrylate. Combinations of these examples are envisioned so as to form various alkyl cyanoacrylate monomer blends.

A second cyanoacrylate monomer component, or blend of components [monomer(s) B], is derived from monomers comprising, based upon total weight of the formulation, approximately 0 to 90 weight percent. The B monomer is selected from at least one alkyl radical containing approximately 6 to 10 carbon atoms in the alkyl group. Representative examples of an alkyl cycanoacrylate monomer include hexyl, heptyl, octyl, nonyl, and decyl cyanoacrylate. Combinations of these examples are envisioned so as to form various alkyl cyanoacrylate monomer blends.

Plasticizer agents suitable for providing optimum flexibility are incorporated into the adherence component composition. In addition, stabilizer agents suitable to provide the fluid composition with increased stability are incorporated into the adherence component composition. In addition, viscosity enhancing agents suitable to provide the fluid composition with increased viscosity are incorporated into the adherence component composition.

### DESCRIPTION OF THE PREFERRED EMBODIMENT

### 1. Detailed Description

The advantages and features of the present invention will become better understood with reference to the following more detailed description and claims. The present invention provides a fluid composition comprised of a coatable composition adapted to form, in situ, a protective superimposing element, seal or covering element. The fluid composition is in the form of a liquid, viscous liquid, or the like. The fluid composition is adapted to be coated onto a substrate using any convenient coating technique, including by means of spraying, aerosol, pump, brushing, swabbing, direct coating, or combinations of these or the like. The fluid composition is adapted to form a protective or preventative covering or bandage for superficial minor cuts, scrapes, abrasions, burns and the like. The fluid composition is further adapted to form a seal on non-superficial tissues or to close open tissues exceeding minor cuts, scrapes and abrasions.

The coatable composition is defined of an effective amount of an adherence component in order that the covering element adheres to the desired substrate. Further, an effective amount of adherence component prevents an exposed surface of resultant covering element from being undesirably tacky resulting in unfavorable difficulty after coating the fluid composition onto the desired substrate when employing the desired coating technique.

A variety of adherent materials, which form a tacky material in situ, may be used as the adherence component. The adherence component should readily adhere to the treatment site of the substrate over which the covering element will form. Additionally, the adherence component is adapted to be substrate compatible in order to avoid unreasonable irritation at treatment site. Further, the adherence component is sufficiently flexible to facilitate its conformation with the contours of the treatment site without drying, cracking, and causing undue restriction of substrate movement.

A particularly beneficial, as well as advantageous, class of adherent materials satisfying these criteria is the class of cyanoacrylate monomers, preferably a blend of cyanoacrylate monomers and synergistic components. The ultimate formulation and corresponding weight percentages of cyanoacrylate monomers and synergistic components are established based on the intended application. Varying these percentages is the method allowing for adjustability in set time, exothermic heating upon monomer polymerization, stability, viscosity and flexibility of adherence component, as opposed to the same attributes exhibited by other systems such as methacrylate polymers and acrylate embodiments thereof.

A preferred cyanoacrylate monomer formulation is derived from monomers comprising, based upon total weight of the formulation, approximately 35 to 100, and preferably 10 to 90 percent of the total weight is (A) cyanoacrylate monomers. The A monomer is selected from at least one alkyl radical containing approximately 1 to 5 carbon atoms in the alkyl group. Representative examples of A alkyl cycanoacrylate monomers include methyl, ethyl, n-butyl, isobutyl, n-pentyl, 2-pentyl or 3-pentyl cyanoacrylate. Preferably, the A alkyl cyanoacrylate is selected from butyl cyanoacrylate. Combinations of these examples are envisioned so as to form various alkyl cyanoacrylate monomer blends.

Preferably 10 to 90 percent of the total weight is (B) cyanoacrylate monomers. The B monomer is selected from at least one alkyl radical containing approximately 6 to 10 carbon atoms in the alkyl group. Representative examples of B alkyl cycanoacrylate monomers include hexyl, heptyl, octyl, nonyl, and decyl cyanoacrylate. Preferably, the B alkyl cyanoacrylate is selected from octyl cyanoacrylate. Combinations of these examples are envisioned so as to form various alkyl cyanoacrylate monomer blends.

Plasticizer agents suitable for providing optimum flexibility are incorporated into the formulation composition. The plasticizer component composition incorporates from approximately 0 to 65 percent of total fluid composition formulation weight.

Stabilizer agents suitable for providing optimum stability are incorporated into the formulation composition. The stabilizer component composition incorporates from approximately 0 to 65 percent of total fluid composition formulation weight.

Viscosity enhancing agents suitable for providing optimum viscosity are incorporated into the formulation composition. The viscosity enhancer component composition incorporates from approximately 0 to 65 percent of total fluid composition formulation weight.

Another preferred cyanoacrylate monomer formulation is derived from monomers comprising, based upon total weight of the formulation, approximately 35 to 100, and preferably 70 to 100 percent of the total weight is (A) cyanoacrylate monomers. The A monomer is selected from at least one alkyl radical containing approximately 1 to 5 carbon atoms in the alkyl group. Representative examples of A alkyl cycanoacrylate monomers include methyl, ethyl, n-butyl, isobutyl, n-pentyl, 2-pentyl or 3-pentyl cyanoacrylate. Preferably, the A alkyl cyanoacrylate is selected from butyl cyanoacrylate. Combinations of these examples are envisioned so as to form various alkyl cyanoacrylate monomer blends.

Preferably 0 to 30 percent of the total weight is (B) cyanoacrylate monomers. The B monomer is selected from at least one alkyl radical containing approximately 6 to 10 carbon atoms in the alkyl group. Representative examples of B alkyl cycanoacrylate monomers include hexyl, heptyl, octyl, nonyl, and decyl cyanoacrylate. Preferably, the B alkyl cyanoacrylate is selected from octyl cyanoacrylate. Combinations of these examples are envisioned so as to form various alkyl cyanoacrylate monomer blends.

Plasticizer agents suitable for providing optimum flexibility are incorporated into the formulation composition. The plasticizer component composition incorporates from approximately 0 to 65 percent of total fluid composition formulation weight.

Stabilizer agents suitable for providing optimum stability are incorporated into the formulation composition. The stabilizer component composition incorporates from approximately 0 to 65 percent of total fluid composition formulation weight.

Viscosity enhancing agents suitable for providing optimum viscosity are incorporated into the formulation composition. The viscosity enhancer component composition incorporates from approximately 0 to 65 percent of total fluid composition formulation weight.

Selected cyanoacrylate monomer formulations are commercially available and dispensed from a multiple application, single container. Additionally, selected cyanoacrylate monomer formulations are commercially available and dispensed from a blown, filled and sealed container for single or multiple applications.

### 2. Operation of the Preferred Embodiment

To use the present invention, the fluid composition is applied using any convenient coating technique to a substrate which forms, in situ, a protective, seal or covering element which functions as a liquid bandage, skin protectant, tissue sealant or skin closure.

Therefore, the foregoing description is included to illustrate the operation of the preferred embodiment and is not meant to limit the scope of the invention. As one can envision, an individual skilled in the relevant art, in conjunction with the present teachings, would be capable of incorporating many minor modifications that are anticipated within this disclosure. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be broadly limited only by the following Claims.

What is claimed is:

1. A fluid composition adapted to form a protective or preventative covering or bandage for superficial minor cuts, scrapes, abrasions, burns and comparable wounds comprising:
a 10-100% by final composition, fluid monomer selected from at least one cyanoacrylate derivative of approximately 1 to 5 carbon atoms in the alkyl branch; and
a 0-90% by final composition, second fluid cyanoacrylate monomer component selected from at least one derivative of approximately 6 to 10 carbon atoms in the alkyl branch, thereby producing a cyanoacrylate monomer formulation.

2. The fluid composition of claim 1, wherein said formulation contains 0-70% by weight a plasticizer or softening agent.

3. The fluid composition of claim 1, wherein said formulation contains 0-50% by weight a viscosity enhancer.

4. The fluid composition of claim 1, wherein said formulation contains 0-30% by weight a monomer stabilizer.

5. The fluid composition of claim 1, wherein said formulation is commercially available and dispensed from a multiple application, singe container.

6. The fluid composition of claim 1, wherein said formulation is commercially available and dispensed from a blown, filled and sealed container for single or multiple applications.

7. A fluid composition adapted to form a seal on non-superficial tissues or to close open tissues exceeding minor cuts, scrapes and abrasions comprising:
a 10-100% by final composition, fluid monomer selected from at least one cyanoacrylate derivative of approximately 1 to 5 carbon atoms in the alkyl branch; and
a 0-90% by final composition, second fluid cyanoacrylate monomer component selected from at least one derivative of approximately 6 to 10 carbon atoms in the alkyl branch, thereby producing a cyanoacrylate monomer formulation.

8. The fluid composition of claim 7, wherein said formulation contains 0-70% by weight a plasticizer or softening agent.

9. The fluid composition of claim 7, wherein said formulation contains 0-50% by weight a viscosity enhancer.

10. The fluid composition of claim 7, wherein said formulation contains 0-30% by weight a monomer stabilizer.

11. The fluid composition of claim 7, wherein said formulation is commercially available and dispensed from a multiple application, singe container.

12. The fluid composition of claim 7, wherein said formulation is commercially available and dispensed from a blown, filled and sealed container for single or multiple applications.
